(19) United States
(12) Patent Application Publication
(10) Pub. No.: US 2006/0286566 A1
(43) Pub. Date: Dec. 21, 2006

(54) Detecting apparent mutations in nucleic acid sequences

(75) Inventors: Stanley N. Lapidus, Bedford, NH (US); Howard Weiss, Newton, MA (US)

Correspondence Address: EDWARDS & ANGELL, LLP, P.O. BOX 55874, BOSTON, MA, 02205, US

(73) Assignee: Helicos BioSciences Corporation, Cambridge, MA (US)

(21) Appl. No.: 11/347,350

(22) Filed: Feb. 3, 2006

Related U.S. Application Data
(60) Provisional application No. 60/649,879, filed on Feb. 3, 2005.

(51) Int. Cl. C12Q 1/68; G06F 19/00

(52) U.S. Cl. 435/6; 702/20

(57) ABSTRACT

A target nucleic acid sequence information obtained from a biological sample can be compared against a collection of reference nucleic acid sequences. The target nucleic acid sequence is aligned or matched against the reference sequences, wherein some of the target sequences have one or more polymorphisms. Different collections of reference sequences are created and used depending on what one is trying to determine about the target. For example, reference sequences associated with a particular disease may be stored in one or more databases and subsequently compared with a target sequence to determine whether a patient from which the sample sequence was obtained has that disease.

### CROSS-REFERENCE TO RELATED CASE

This claims priority to and the benefit of Provisional U.S. Patent Application Ser. No. 60/649,879, filed Feb. 3, 2005, the entirety of which is incorporated herein by reference.

### TECHNICAL FIELD

The disclosed technology generally relates to nucleic acid sequences and, more particularly, to identifying unique, non-repeating segments of nucleic acid sequences with reference to a known or standard human genome.

### BACKGROUND INFORMATION

Completion of the human genome has paved the way for important insights into biologic structure and function. Knowledge of the human genome has given rise to inquiry into individual differences, as well as differences within an individual, as the basis for differences in biological function and dysfunction. For example, single nucleotide differences between individuals, called single nucleotide polymorphisms (SNPs), are responsible for dramatic phenotypic differences. Those differences can be outward expressions of phenotype or can involve the likelihood that an individual will get a specific disease or how that individual will respond to treatment. Moreover, subtle genomic changes have been shown to be responsible for the manifestation of genetic diseases, such as cancer. A true understanding of the complexities in either normal or abnormal function will require large amounts of specific sequence information.

Relatively recent advancements in bioinformatics and genomic research have improved our understanding of how genes and their expressions affect health or disease states. For example, quantitative determination and classification of nucleic acid expression in tissues of interest have been instrumental in identifying correlations between complex disorders, such as cancer, altered expressions and defects in genes. The aggregate knowledge gleaned from such known correlations, coupled with the speed at which new correlations are identified, directly affect a health practitioner's ability to provide an early diagnosis and potential treatment for diseased states.

Various approaches to such nucleic acid sequencing exist. One conventional way to do bulk sequencing is by chain termination and gel separation, essentially as described by Sanger et al., Proc. Natl. Acad. Sci., 74(12): 5463-67 (1977). That method relies on the generation of a mixed population of nucleic acid fragments representing terminations at each base in a sequence. The fragments are then run on an electrophoretic gel and the sequence is revealed by the order of fragments in the gel. Another conventional bulk sequencing method relies on chemical degradation of nucleic acid fragments. See, Maxam et al., Proc. Natl. Acad. Sci., 74: 560-564 (1977). Finally, methods have been developed based upon sequencing by hybridization. See, e.g., Drmanac, et al., Nature Biotech., 16: 54-58 (1998).

Existing sequencing techniques for determining and classifying nucleic acid sequences for all or most of an organism's genes are not optimal when processing the large quantity of sequence data involved. The computational burden and corresponding processing time experienced by such sequencing techniques are further adversely impacted when applied to subtle genetic alterations, such as genetic polymorphisms (e.g., mutations).

Genetic polymorphisms can manifest themselves in several forms, such as point mutations where a single base is changed to one of the three other bases, deletions where one or more bases are removed from a nucleic acid sequence and the bases flanking the deleted sequence are directly linked to each other, and insertions where new bases are inserted at a particular point in a nucleic acid sequence adding additional length to the overall sequence. Large insertions and deletions, often the result of chromosomal recombination and rearrangement events, can lead to partial or complete loss of a gene. Of these forms of mutation, a difficult type of mutation to screen for and detect is the point mutation, because the point mutation represents the smallest degree of molecular change. Detection of all of the polymorphisms associated with a single gene, whether at the genomic level or simply for the entire pools of exons that comprise that gene, remains impractical in research or diagnostic applications owing to the high cost and lengthy processing times of sub-cloning and Sanger sequencing used by conventional techniques. Although existing alignment algorithms are available, such algorithms use suffix trees and some form of maximal subsequence matching. Those algorithms typically require execution times that are unacceptably long for high-throughput methods.

### SUMMARY OF THE INVENTION

Genomic researchers, bioinformatic professionals, healthcare practitioners, and other entities have a continuing interest in developing and using techniques that can identify polymorphisms, differences between a known sequence and a sample being analyzed (hereinafter a “target sequence” or a “sample sequence”), and other useful information from genomic data in a manner that significantly reduces the processing time and cost of such investigations.

The disclosed technology provides systems, algorithms, software, and methods for rapidly compiling the sequence and placement in the genome of DNA and/or RNA. The invention is especially useful in connection with single molecule sequencing methods in which the sequence of individual nucleic acid strands is obtained one molecule at a time in order. Single molecule sequencing techniques result in a sequence that is specific to an individual or to a discrete region of the genome or transcriptome of an individual, thus allowing elucidation of individual differences in sequence. Those individual differences are then correlated to phenotype. The disclosed technology allows the rapid compilation of sequencing data, and is applicable to bulk sequencing and single molecule sequencing alike but has particular application in high-throughput sequencing such as that employed in single molecule techniques.

The disclosed technology involves capturing polymorphisms related to a known reference sequence and appropriately marking the polymorphisms of the target sequence being analyzed. In one illustrative embodiment, the disclosed technology can be used to develop systems and perform methods in which polymorphisms are indicative of certain ailments, conditions, tendencies, and the like. The polymorphisms are identified quickly by analysis of target sequences with respect to known reference sequences, past samples, and the like.

In one embodiment, the disclosed technology is directed to a method of detecting an apparent mutation in a target nucleic acid sequence. The method includes providing a first plurality of sequence segments associated with a reference nucleic acid sequence, each of the first plurality of sequence segments being unique relative to one another. A second plurality of sequence segments corresponds to possible variations in the first plurality of sequence segments. This method compares a portion of the target nucleic acid sequence with the second plurality of sequence segments to detect a match for that portion of the target nucleic acid sequence. If a match is not found, the method continues by comparing the portion of the target nucleic acid sequence with the second plurality of sequence segments to detect a variation in the target nucleic acid sequence.

In a further embodiment, each of the first plurality of sequence segments is between about 15 and 100 bases in length, the second plurality of sequence segments is limited to single-base mutations, additions, and deletions. The reference nucleic acid sequence may correspond to one of a genomic DNA sequence, a cDNA sequence, an RNA sequence, a cancer genome, a developmental gene, an infectious agent, or an inherited gene. It is also possible that the variation corresponds to a sequencing error in the target nucleic acid sequence, a difference between organisms of a common type, a time-based difference in an organism, a post-treatment difference in an organism, or a disease condition state. Preferably, the second plurality of sequence segments are sorted to facilitate the comparison with the portion of the target nucleic acid sequence.

In another embodiment, the disclosed technology is directed to a method of forming a data repository of sequence segments to facilitate detection of apparent mutations in a target nucleic acid sequence. The method includes the steps of accessing a first plurality of sequence segments associated with a reference nucleic acid sequence, each of the first plurality of sequence segments being unique relative to one another, determining possible variations for at least some of the first plurality of sequence segments and storing the possible variations in the data repository for subsequent comparison with at least a portion of the target nucleic acid sequence to detect apparent mutations therein. To reduce the storage needs, a subset of the stored variations may be removed from the data repository based on an inability to occur within an organism associated with the target nucleic acid sequence. Further, the method may store genomic locations associated with the first plurality of sequence segments in the data repository and associate each of the stored genomic locations with at least some of the stored possible variations. Still further, the method may associate a genomic location of each of the first plurality of sequence segments with corresponding possible variations.

In still another embodiment, the disclosed technology is directed to a method of forming a database of G-tag k-mers of a reference DNA including the steps of assembling a list of consensus G-tag k-mers and adding naturally-occurring single-variant G-tag k-mers to the list. This method may also include the steps of adding naturally-occurring dual-variant G-tag k-mers to the list, ordering the list alphabetically or limiting the list to one strand of the reference DNA. Preferably, the naturally-occuring single-variant G-tag kmers are associated with a particular disease. In a further aspect, the method associates a location in a human genome for each of the list of consensus G-tag k-mers and naturally-occurring single-variant G-tag k-mers.

It should be appreciated that the present invention can be implemented and utilized in numerous ways, including without limitation as a process, an apparatus, a system, a device, a computer, a method for applications now known and later developed or a computer readable medium. These and other unique features of the system disclosed herein will become more readily apparent from the following description and the accompanying drawings.

### BRIEF DESCRIPTION OF THE DRAWINGS

The foregoing discussion will be understood more readily from the following detailed description, when taken in conjunction with the accompanying drawings in which:

FIG. 1 schematically illustrates one exemplary system for collecting and comparing sequence data in accordance with the disclosed technology; and

FIG. 2 is a flowchart illustrating a method for analyzing sequence data in accordance with the disclosed technology.

### DESCRIPTION

Unless otherwise specified, the illustrated embodiments can be understood as providing exemplary features of varying detail of certain embodiments, and therefore, unless otherwise specified, features, components, modules, elements, and/or aspects of the illustrations can be otherwise combined, interconnected, sequenced, separated, interchanged, positioned, and/or rearranged without materially departing from the disclosed systems or methods. Additionally, the shapes and sizes of components are also exemplary and unless otherwise specified, can be altered without materially affecting or limiting the disclosed technology.

In general, the term “substantially” can be construed to indicate a precise relationship, condition, arrangement, orientation, and/or other characteristic as well as deviations thereof, to the extent that such deviations do not materially affect the disclosed technology, methods, and systems.

One or more digital data processing devices can be used in connection with various embodiments of the invention. Such a device generally can be a personal computer, computer workstation (e.g., Sun, HP), laptop computer, server computer, mainframe computer, handheld device (e.g., personal digital assistant, Pocket PC, cellular telephone, etc.), information appliance, or any other type of generic or special-purpose, processor-controlled device capable of receiving, processing, displaying, and/or transmitting digital data. A processor generally is logic circuitry that responds to and processes instructions that drive a digital data processing device and can include, without limitation, a central processing unit, an arithmetic logic unit, an application specific integrated circuit, a task engine, and/or any combinations, arrangements, or multiples thereof.

Software or code generally refers to computer instructions which, when executed on one or more digital data processing devices, cause interactions with operating parameters, sequence data/parameters, database entries, network connection parameters/data, variables, constants, software libraries, and/or any other elements needed for the proper execution of the instructions, within an execution environment in memory of the digital data processing device(s). Those of ordinary skill will recognize that the software and various processes discussed herein are merely exemplary of the functionality performed by the disclosed technology and thus such processes and/or their equivalents may be implemented in commercial embodiments in various combinations and quantities without materially affecting the operation of the disclosed technology.

In brief overview, the disclosed technology relates to comparing target nucleic acid sequence information obtained from a biological sample against a collection of reference nucleic acid sequences. More particularly, the disclosed technology can be used to align or match a set of target sequences, wherein some of the target sequences have one or more polymorphisms. Different collections of reference sequences can be created and used depending on what one is trying to determine about the sample or target sequence(s). For example, reference sequences associated with a particular disease may be stored in one or more databases, tables, and/or other types of data repositories and may be subsequently compared with one or more sample or target sequences to determine whether a patient from which the sample sequences were obtained has that disease. As another example, the set of reference sequences can be every possible combination of k-mer segments (say, 25-mers) whether found in the human genome or not. The disclosed technology can facilitate the formation and/or population of such data repositories, as well as facilitate comparisons involving data stored therein.

In one illustrative embodiment, the disclosed technology is used to develop a compilation or table of alternative sequences that may be present at certain locations on the genome of an organism, thereby allowing the identification of sequence in samples that have mutations or variations due to other sources (e.g., sequencing error) in a computationally reduced manner. In accordance with one aspect of the invention, the reference list may comprise all possible or known naturally occurring 25-mers of a given length in a particular species' genome (e.g., all 25-mers present in the human genome). The database may alternatively contain a subset of genomic DNA or RNA. For example, the database may contain all oncogene sequences of a predetermined length or all messenger RNA sequences of a predetermined length. The length of the sequences may be determined by the complexity of the database and/or the resolution desired in matching a sample sequence against the reference list or table. For example, the longer the individual sequence entries in the database, the fewer matches, on average, are expected between a reference sequence in the database and a sequence derived from a sample.

In general, the number of bases in the sample or target sequence segment is equal to the number of bases in each reference sequence segment in the database. For example, if the target sequence is “ATGCTCATTA”, each of the entries in the database would be ten bases (or letters) in length.

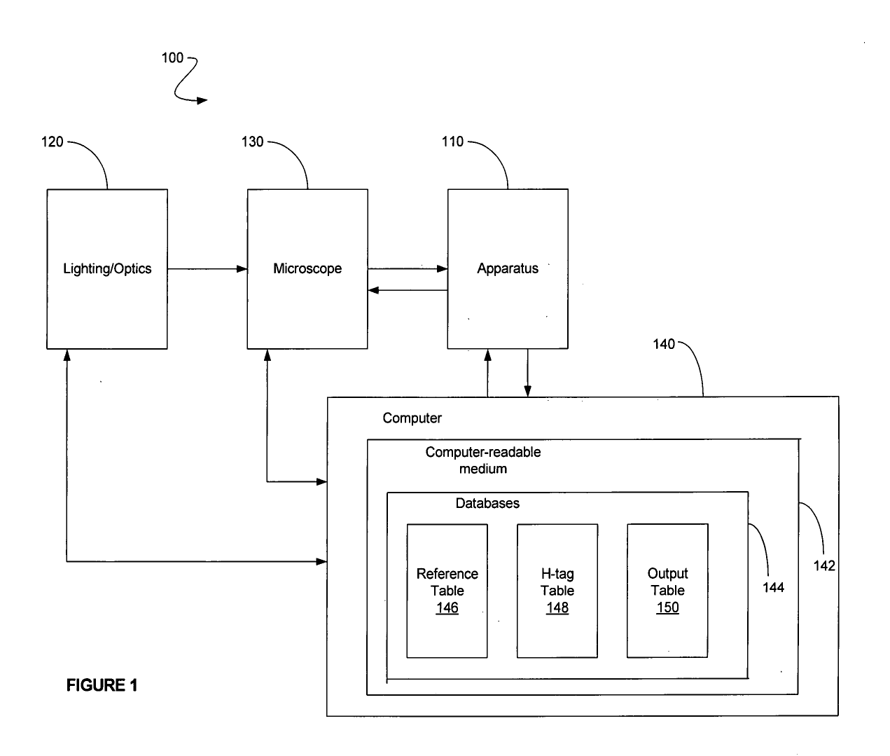

In some embodiments, one or more look-up tables can be used to analyze the results of DNA sequencing methods, particularly for high-throughput sequencing methods. An exemplary system that may be used to perform single-molecule sequencing is shown in FIG. 1. In FIG. 1, a system 100 permits sequencing by synthesis of a nucleic acid from a sample. The system 100 includes an apparatus 110 for handling small fluid volumes and also includes other components including a lighting/optics module 120, a microscope module 130, and a digital data processing device 140. These elements communicate with and/or interrelate to one another generally as shown by the arrows in FIG. 1.

The lighting/optics module 120 can include multiple light sources and filters to provide light to a microscope (not shown) of the microscope module 130 for viewing and analysis. The light is reflected onto a flow cell that has the sample therein or thereon and that is seated near (e.g., above or below) the microscope. The microscope module 130 includes hardware for holding the flow cell and moving a microscope stage and an imaging device. The digital data processing device 140 includes and/or is communicatively coupled to at least one computer-readable medium 142 containing a database area 144. By way of non-limiting example, a computer-readable medium 142 can include a variety of memory types and memory storage devices, such as, for example, one or more volatile memory elements (e.g., random access memory), nonvolatile memory elements (e.g., read only memory, EEPROM, etc.), hard drives, floppy drives, floptical drives, CD-ROMs, DVDs, USB memory sticks, and/or any other type of memory or device, separately or in any combination or multitude, that may be used to store and/or access computer-executable instructions and/or digital data (e.g., database records, nucleic acid sequences, etc.) necessary for the proper operation of the disclosed technology. It is envisioned that the computer readable medium 142 may be distributed among several devices and across large geographic areas, although for simplicity it is shown as a single unit. As is known to those skilled in the art, a digital data processing device 140 can include, without limitation, one or more computer-readable media, processor(s), devices, controllers, user interfaces, software programs, and/or any other computer components necessary for operating the system 100 in accordance with the disclosed technology for storing, accessing, and/or analyzing nucleic acid sequence information.

In one illustrative operation, a nucleic acid from a sample is fragmented and immobilized in a flow cell. The nucleic acid in the flow cell includes a primer binding site to which a complementary primer nucleic acid has hybridized. The apparatus 110 injects into the flow cell a solution comprising a fluorescent nucleotide and a polymerase in a buffered solution under conditions permitting incorporation of the fluorescent nucleotide at the end of the primer, if and only if the fluorescent nucleotide is complementary to the first position of the nucleic acid.

The apparatus 110 then injects a wash solution to remove any unincorporated nucleotides and the lighting/optics module 120 then detects the presence or absence of fluorescence at the location of the nucleic acid, which is recorded by the digital data processing device 140. The fluorescent nucleotide can then be bleached or the fluorescent label is removed and the apparatus 110 injects a different nucleotide/polymerase/buffer solution. The system 100 iterates the process until enough sequence information for a sample of interest has been recorded by the digital data processing device 140 to permit comparison of the recorded sample sequence to the entries in a reference table 146 stored in the database area 144 contained on or in the computer-readable medium 142. The resulting target data is a plurality of target “H-tags” (as defined below) to be aligned (that is, matched) or otherwise processed.

DNA is composed of four basic subunits (bases or nucleotides) that form a linear sequence. It is the sequence in which the subunits occur that provides genetic coding information (e.g., genes). The four bases are adenine, thymine, cytosine, and guanine (in RNA, uracil is substituted for thymine). The human genome is roughly composed of 3 billion bases. For ease of reference, each base is represented by a genome tag (or G-tag) in one preferred embodiment of the invention. The four possible G-tags are represented as A, G, T, and C for adenine, guanine, thymine and cytosine, respectively, of DNA. The reference or concensus human genome can be represented by a single list of approximately 4.5 billion G-tags.

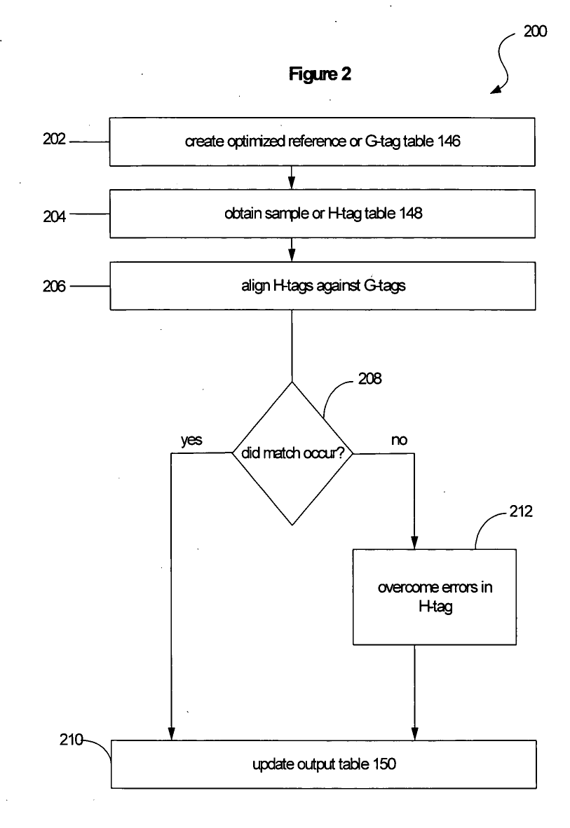

Referring now to FIG. 2, a flowchart 200 depicts a process for facilitating detection of mutations in a target nuclei by comparing a portion of the target nuclei with a sequence of reference segments based upon a consensus human genome. The flowchart 200 illustrates the structure or the logic of a possible embodiment according to the invention for execution on a computer, digital processor, or microprocessor. As such, the flowchart would be rendered in a different form such as computer software code to instruct a digital processing apparatus (e.g., computer) to perform a sequence of function steps corresponding to those shown in the flowchart.

At step 202, the system 100 creates the reference or base table 146 (see FIG. 1). In one embodiment, the consensus human genome is represented by an ordered list of sequence segments in the reference table 146 where each segment is, in this embodiment, 25 base G-tags. Parsing the G-tags of the human genome into k-mers (say, 25-mers) and arranging them into an ordered (say, alphabetical) list of k-mers facilitates searching the reference table 146. A 25-base G-tag can be represented by a number in the range 0-4²⁵ or 0-2⁵⁰ in the reference table 146. Each record in the list may contain additional information including, without limitation, the address or location in the human genome of the respective G-tag and/or a pointer. The pointer can be utilized for resolving mismatches, as described below.

At step 204, a nucleic acid sequence of a sample can be obtained from the system 100 of FIG. 1 and stored in an H-tag table 148 therein. An actual k-base (say, 25-base) read of a sequence of a sample, as measured by the system 100, can be referred to as an H-tag k-mer. The system 100 typically is run multiple times on the same sample (e.g., ten times) to statistically improve the results. A typical experiment would create an ordered list of 1.2 billion H-tags. If 25-base segments are captured, then each base in each of these 25-mer table entries can be referred to as an “H-tag” where “H” is indicative of the assignee, Helicos BioSciences of Cambridge, Mass., for the subject technology.

At step 206, the system 100 aligns or matches the 1.2 billion target H-tags against the reference 4.5 billion G-tags to create an output table 150 showing where each target H-tag lies on the genome backbone. In one embodiment, the H-tag table 148 of target H-tag k-mers and the reference table 146 of G-tag k-mers are sorted in ascending order and the reference G-tag k-mers are searched for a match for each target H-tag k-mer in the target list of H-tag table 148.

At step 208, if a match occurs, the process proceeds to step 210. At step 210, the location of the respective target H-tag k-mer is added to that record in the output table 150. The process continues by selecting an additional target H-tag k-mer and repeating until the entire set of k-mers in the H-tag table 148 has been processed. In one comparison method, a binary search against an index is used to speed up searching for a match. In another embodiment, a paged memory scheme is further utilized to increase computational efficiency. Binary searching may be advantageously modified to correlate a starting point of location in the H-tag table 148 with the starting point in the reference table 146.

On the other hand, if there is no match at step 208, the process proceeds to step 212. At step 212, the system 100 has challenges in completing the target list of output table 150. A mismatch can be biological in which the sequence of the target genome contains biological polymorphisms such as insertions (extra bases), deletions (missing bases), or mutations (substitution of one base for another). A mismatch also can occur as a result of instrument error such as the system 100 not recording a base that was actually present in a sample, detecting an extra base that is not actually present in a sample, or an incorrect identification of a base in the sample (e.g., dectecting a “T” as a “G”). Deletions are the most common error.

In one embodiment, the system 100 overcomes errors by performing a “best” or closest match alignment, allowing for errors in the sequencing of the target material and differences between the sequenced target genetic material and the reference sequence segments. Erroneous sequences should produce single instances of mismatched alignments whereas differences between the sequenced genetic material and the reference genome should produce multiple mismatched alignments. Assuming an error rate of approximately 4%, approximately 36% of the generated sequences will be error free, 37% will have a single error, and 17% of the sequences will have 2 errors. Hence, if the target sequences can be aligned, more than 90% of the target sequences generated by the system 100 to predict the composition of the sequenced target genetic material will be correct.

One challenge to the system 100 is in creating the reference table 146. There are approximately 3 billion “letters” (i.e., bases) in the human genome. Hence, there are approx 6 billion 25-mers, considering sequences on both strands of the DNA. This is out of a possible 4²⁵ or approx 1.12×10¹⁵ possible 25-letter “words” constructed from the 4 letters A, C, T, and G. Hence, only approximately 1 in every 2×10⁵ possible sequences is a real sequence. Although it is reasonable to create a list or catalog of all of the possible sequences, it is a larger exercise to use some mechanisms (such as a bit map) to indicate the existence of a given sequence in the genome. For example, an analysis can include sequences generated from both strands of the DNA of the target material. However, by virtue of the two DNA strands being reverse complement (i.e., A always pairs with T, and G with C), an optimization of the reference table 146 is to only store one strand, i.e., perform the analysis on only one strand of the reference DNA. After the target sequences are found, not only are the target sequences searched but both the forward and reverse complement of each found sequence is searched.

Further, many of the 25-mers in the consensus genome occur multiple times. In other words, the same H-tag k-mer exists at different places in the human genome. It is believed that approximately 20% of the genome is covered by repeated sequence. Thus, a single 25-mer entry can simply be associated by a pointer with the various positions of occurrence to shorten the reference table 146. Preferably, all of the possible found locations are marked with a fractional probability of their location in the reference table 146.

Another preferred approach is to divide the underlying reference genome in reference table 146 into segments which can be mapped uniquely and sections which are repeated. The repeat count is of interest to the genomics community. The frequency with which repeated sequences are found can be used to predict the frequency with which repeated genetic material occurs in the genome.

In another embodiment, the reference table 146 includes all single or perhaps even double and triple (and beyond) error variants of the sequences. For each error free sequence, there are 125 error variants, created by deletions, insertions, and single-base substitutions. This expands the catalog from 6 billion to 750 billion, a large number but still small in comparison to 1.12×10¹⁵ and well within the capacity of terabyte or petabyte rotating memory systems. By simple extrapolation, it would take 150×20 minutes or 3000 minutes to perform the comparison using a currently-available, off-the-shelf computer system.

If the reference table 146 contains all the two-error variants of each sequence, the reference table 146 would become exceedingly large for most generally-available storage systems. There are several possible approaches to overcoming this challenge to the system 100. For example, an initial match can be performed that separates the sequences into those sequences which match (single or one-error sequences) and those sequences which do not. Subsequently, the process would only need to generate the single-error variants of all the non-matching sequences, sort, and match these sequences. If any of these sequences is a two-error variant of a possible sequence, then some of its variants will “fix” the error and match a one-error variant of one of the possible sequences. A catalog of all the two-error variants would be again 125 times the size of the one-error catalog. The number of entries in this catalog is still small compared to the number of possible 25-mers, but it is large compared to any generally-available storage system and would take an excessive amount of time to peruse. Hence, an advantageous system and method for managing and searching the catalog of sequences alleviates the computational burden. In one embodiment, a mechanism to generate the error catalog “on the fly” overcomes the storage and search challenge as described below. By “on the fly”, the system 100 dynamically computes the error sequences as needed rather than creating the error sequences in advance and storing them.

For the matching to work more efficiently, it is preferable that both the list of sequences in sample table 148 and the reference table 146 are sorted. Given the relative distance between sequential entries in the reference table 146, it is likely that error variants where the error occurs at the end of the word would still be positioned between sequential entries in the catalog. However, this would not be true for errors at the beginning of a sequence and the matching should accommodate these variants.

In one embodiment, the process 200 constructs a lookup table of the found sequences and then searches for the two-error variants of the genomic sequences in that table, where the two-error variants are generated as needed rather than compiled in advance, i.e., on the fly. In one embodiment, a special-purpose computer or a software program executes on a general-purpose computer that holds all the found sequences (or only the found sequences which failed to match a catalog of zero- and one-error variants of the possible sequences) and performs the match since the memory requirement to hold the list of found sequences is limited (if 16 bytes per found sequence are allowed—7 bytes to code the sequence value and 9 bytes to store location information and other properties—the required memory is still only 16×1.2 billion=20 Gb).

As noted above, one alternative to a straightforward comparison of an ordered list of found tags to an ordered list of sequences and variations is to use on the fly computation of variants. On the fly computation becomes increasingly feasible as the length of the ordered list increases and/or the length of the tag becomes shorter. Preferably, the sequences are ordered by the genomic alphabet. Hence, if any two sequential sequences in the sorted list are considered, it is likely that a significant number of possible sequences fit between them on the human genome. In other words, the two sequential sequences likely span a significant range. A distance between any two sequences is defined consistent with the ordering. For instance, a 25-mer can be expressed as 50 bit number, using 2 bits to encode each base (A==0, C==1, G==2, T==3). A sequence of 25 A's is represented by 0, a sequence of 25 T's is represented as 2ˆ50−1. Other sequence equivalents are

1 = AAAAAAAAAAAAAAAAAAAAAAAAC
2 = AAAAAAAAAAAAAAAAAAAAAAAAG
3 = AAAAAAAAAAAAAAAAAAAAAAAAT

This representation of each sequence as a 50 bit number defines a distance between two sequences consistent with an alphabetical order.

Consider the single error variants of any sequence where the error variants are generated as described above. For simplicity, the following description does not change the length of the sequence. If the original sequence is

CCCCCCCCCCCCCCCCCCCCCCCCC
(equivalent to 1555555555555 in Hexadecimal
notation)

then the tail end variants are

Hex 1555555555554 = CCCCCCCCCCCCCCCCCCCCCCCCA
Hex 1555555555556 = CCCCCCCCCCCCCCCCCCCCCCCCG
Hex 1555555555557 = CCCCCCCCCCCCCCCCCCCCCCCCT

which are quite near to one another. Hence, during matching, the process 200 examines a buffer which contains the sequence

Hex 1555555555555 = CCCCCCCCCCCCCCCCCCCCCCCCC

then it will also contain the sequences

Hex 1555555555554 = CCCCCCCCCCCCCCCCCCCCCCCCA
Hex 1555555555556 = CCCCCCCCCCCCCCCCCCCCCCCCG
Hex 1555555555557 = CCCCCCCCCCCCCCCCCCCCCCCCT

Therefore, without changing the buffer, the process 200 creates the 25-th base substitution variants on the fly and compares these variants to the reference. The same logic applies as error location moves from the 25-th position to the 24-th position to the 23-rd position and so on.

If the density of found sequences is approx 4ˆ25/(6*10ˆ9) or approximately one every 200,000 positions, it likely that searching between any two sequences in the list for all variations up to 4ˆ9=262,144 or for variations in the last 9 positions of the sequence can be accomplished efficiently. In one embodiment, the system 100 buffers 4ˆ15=1073741824 (1 billion) genomic sequences in memory. Thus, all substitution variants in 24=9+15 base sequences can be easily searched. The process 200 reads in candidate tags, generates the substitutions, sorts the list and then compares the candidate tags against the portion of the sorted list of genomic tags currently held in the computer memory until one or more matches are found. Turning to 25-mers for example, searching for variants caused by substitutions and deletions in the first base in the genomic sequence can be problematic. However, the list of tags is pre-expanded to include those tag variants which arise from substitutions in the first base. As a result, the search time is increased by a factor of five since there are three alternate bases for each string (substitution) plus a deletion.

In another embodiment, due to the sparseness of the found or genomic sequences in the space of possible sequences, a two-stage lookup table could be used to store the sequence data efficiently. A 25-mer can be uniquely encoded as 50-bit sequence. Divide the sequence into a 32-bit “index” and a 20-bit value. One would locate an entry by constructing an “index” table. Each entry in the index table would point to a list of the 20-bit values which actually existed for that index. To lookup a sequence, one would convert the sequence to a 50-bit word, divide the sequence into its index and value fields, locate the corresponding value list in the index table, and then match the value portion against the corresponding value list. The actual lengths of the index and value fields could be optimized to minimize the memory requirement (e.g., fewest empty entries in the index portion of the table) or the lookup time (e.g., fewest entries in the value chains). Alternatively, a hashing function could be designed to optimize one or both parameters.

In still another alternative embodiment, at step 206 the process 200 uses a matching algorithm to find the maximal match for a given sequence in a list of possible matching sequences. This algorithm is based on the observation that nearly all 25-mers will include at least a smaller subsequence, such as a 13-mer subsequence, which is error free. One can construct a two-stage lookup table, where the index portion of the table is the list of possible 13-mers, and the value portion of the table is the possible “suffices” of that 13-mer in the human genome. For a given sequence, one takes the initial 13 letters, and looks up that 13-mer in the index table. Then, the system 100 determines how many of the subsequent remaining 12 letters match the suffix for that 13-mer to yield a candidate maximal match.

Based upon this maximal match, the system 100 generates a new 13-mer to lookup and a new suffix to match, where the new suffix is only 11 letters. If the resulting match is longer than the previous match, this becomes the new candidate maximal match. The system 100 continues until it is no longer possible to find a longer candidate maximal match. As a result, the system 100 requires less computational processing and storage to arrive at a result.

In another embodiment, the process identifies a reference sequence segment (say, a 25-mer) that best matches the sample H-tag k-mer (also a 25-mer, say) even if the two are not identical. A match can be selected, for example, by identifying a particular original reference nucleic acid sequence that best corresponds (e.g., exhibits a greater amount of matching nucleotides) to an original sample nucleic acid sequence that was obtained from a DNA sequencing reaction.

Specifically, for each of the original reference nucleic acid sequences, the probability of sequencing errors yielding the observed original nucleic acid sequence from the sample can be calculated. The probability can be based, at least partly, on the sequencing method and conditions encountered and may be based on empirical observations and/or theoretical calculations. The original reference nucleic acid sequence of highest probability is selected as the matching sequence. One exemplary way of determining the likelihood that one of a set of matching reference sequences is the correct sequence involves the use of Bayes theorem and probability concepts to arrive at an equation that yields a probability value for each candidate matching reference sequence as follows:
P(Sᵢ)=Omegaᵢ/(the sum over k of Omegaₖ)

In this equation, k and the subscript i go from 1 to n, n being a positive integer, Sᵢ represents each of the n candidate matching reference sequences, and Omega represents the a priori probability of the sequencing machine generating the observed sequence using the measured sample and parameters. In another embodiment, the disclosed technology overcomes these errors by finding similar reference k-mers to the erroneous or mutated target H-tag k-mer, comparing the subject target H-tag k-mer to an ancillary list in order to find an alternative match and marking the disparity in the target list. In still another embodiment, once the system 100 identifies the best match for the target H-tag k-mer, the target H-tag k-mer is compared to an alternative table, which is a portion of the reference table 146. A pointer of the record for the best match identifies the location of the alternative table for that respective reference sequence segment. The alternative table may include typical variations such as known mutations, common erroneous readings, and the like. If a match is found for the target H-tag k-mer in the alternative table, the system 100 notes the disparity and inserts the likely location on the reference human genome into the output table 150.

In one embodiment, the alternative table is limited to single-base mutations, additions, and deletions. The alternative table could include two-base mutations, additions, and/or deletions, or even three-base mutations, additions, and/or deletions, or even beyond. In the single-base situation, the possible patterns of interest for all the reference sequence segments (say, 25-mers) would thus be approximately 660 billion (151×4.2 billion). By storing a pair of numbers representing the pattern (50 bits) and the genome address (32 bits), the entire storage requirement would be on the order of 7.25 tera bytes (660 billion×11). Once the output table 150 is complete, the output table 150 includes records incorporating each target nucleic acid sequence, indication of the matched or most likely location on the consensus genome, and, for mismatched H-tag k-mers, indication of the corresponding mutation or error.

It will be appreciated by those of ordinary skill in the pertinent art that the functions of several elements may, in alternative embodiments, be carried out by more or fewer elements, or a single element. Similarly, in some embodiments, any functional element may perform fewer, or different, operations than those described with respect to the illustrated embodiment. Also, functional elements (e.g., modules, databases, interfaces, computers, servers and the like) shown as distinct for purposes of illustration may be incorporated within other functional elements in a particular implementation.

While the invention has been described with respect to certain illustrative embodiments, various changes and/or modifications can be made without departing from the spirit or scope of the invention. The invention is not limited to or by the particular embodiments disclosed herein.

What is claimed is:

1. A method of detecting an apparent mutation in a target nucleic acid sequence, the method comprising:
a) providing a first plurality of sequence segments associated with a reference nucleic acid sequence, each of the first plurality of sequence segments being unique relative to one another;
b) providing a second plurality of sequence segments corresponding to at least some possible variations in the first plurality of sequence segments;
c) comparing at least a portion of the target nucleic acid sequence with the second plurality of sequence segments to detect a match for the at least a portion of the target nucleic acid sequence; and
d) if the match is not found, comparing the at least a portion of the target nucleic acid sequence with the second plurality of sequence segments to detect a variation in the target nucleic acid sequence.

2. The method of claim 1, wherein each of the first plurality of sequence segments is between about 15 and 100 bases in length.

3. The method of claim 1, wherein second plurality of sequence segments is limited to single-base mutations, additions, and deletions.

4. The method of claim 1, wherein the reference nucleic acid sequence corresponds to at least one of a genomic DNA sequence, a cDNA sequence, an RNA sequence, a cancer genome, a developmental gene, an infectious agent, and an inherited gene.

5. The method of claim 1, wherein the variation corresponds to a sequencing error in the target nucleic acid sequence.

6. The method of claim 1, wherein the variation corresponds to at least one of a difference between organisms of a common type, a time-based difference in an organism, a post-treatment difference in an organism, and a disease condition state.

7. The method of claim 1, further comprising sorting the second plurality of sequence segments to facilitate the comparison with the at least a portion of the target nucleic acid sequence.

8. A method of forming a data repository of sequence segments to facilitate detection of apparent mutations in a target nucleic acid sequence, the method comprising:
accessing a first plurality of sequence segments associated with a reference nucleic acid sequence, each of the first plurality of sequence segments being unique relative to one another;
determining possible variations for at least some of the first plurality of sequence segments; and
storing the possible variations in the data repository for subsequent comparison with at least a portion of the target nucleic acid sequence to detect apparent mutations therein.

9. The method of claim 8, wherein each of the first plurality of sequence segments is about 25 bases in length.

10. The method of claim 8, wherein at least some of the first plurality of sequence segments are of different length.

11. The method of claim 8, further comprising removing a subset of the stored variations from the data repository based on an inability to occur within an organism associated with the target nucleic acid sequence.

12. The method of claim 8, further comprising:
storing genomic locations associated with the first plurality of sequence segments in the data repository; and
associating each of the stored genomic locations with at least some of the stored possible variations.

13. The method of claim 8, further comprising associating a genomic location of each of the first plurality of sequence segments with corresponding possible variations.

14. The method of claim 8, further comprising sorting the stored possible variations to facilitate detection of the apparent mutations.

15. A method of analyzing a target sequence, the method comprising the steps of:
providing a reference nucleic acid sequence, the reference nucleic acid sequence having a plurality of reference sequence segments;
providing a plurality of polymorphic sequence segments corresponding to at least one reference sequence segment;
determining if a target sequence segment of the target nucleic acid sequence is similar to the at least one reference sequence segment; and
if the target sequence segment is similar, comparing the target sequence segment of the target nucleic acid sequence with the plurality of polymorphic sequence segments to detect a polymorphism in the target sequence segment.

16. A method of forming a database of G-tag k-mers of a reference DNA comprising the steps of:
assembling a list of consensus G-tag k-mers; and
adding naturally-occurring single-variant G-tag k-mers to the list.

17. The method of claim 16, further comprising the step of adding naturally-occurring dual-variant G-tag k-mers to the list.

18. The method of claim 16, further comprising the step of ordering the list alphabetically.

19. The method of claim 16, further comprising the step of limiting the list to one strand of the reference DNA.

20. The method of claim 16, wherein the naturally-occuring single-variant G-tag kmers are associated with a particular disease.

21. The method of claim 16, further comprising the step of associating a location in a human genome for each of the list of consensus G-tag k-mers and naturally-occurring single-variant G-tag k-mers.

22. A method of analyzing a target nucleic acid sequence, the method comprising the steps of:
providing a reference nucleic acid sequence, the reference nucleic acid sequence having a plurality of reference sequence segments;
determining if a target sequence segment of the target nucleic acid sequence matches one of the plurality of reference sequence segments;
if the target sequence segment does not match, identifying the target sequence segment as a non-matched target sequence segment;
generating at least one single error variant of the non-matched target sequence segment; and
comparing the at least one single error variant with the plurality of reference sequence segments for a match.

23. A method as recited in claim 22, wherein the at least on single error variant is a selected from the group consisting of a deletion, a mutation, and an insertion.

24. A method as recited in claim 22, further comprising the steps of:
if the at least one single error variant does not match, generating a double error variant of the non-matched target sequence segment; and
comparing the double error variant with the plurality of reference sequence segments for a match.

25. A method of detecting an apparent mutation in a target nucleic acid sequence, the method comprising:
a) providing a first plurality of sequence segments associated with a reference nucleic acid sequence, each of the first plurality of sequence segments being unique relative to one another;
b) comparing at least a portion of the target nucleic acid sequence with the first plurality of sequence segments to detect a match for the at least a portion of the target nucleic acid sequence;
c) if the match is not found, computing a second plurality of sequence segments corresponding to at least some possible variations of the at least a portion of the target nucleic acid sequence; and
d) comparing the at least a portion of the target nucleic acid sequence with the at least some possible variations to detect a variation in the target nucleic acid sequence.

26. The method of claim 25, wherein at least some of the first plurality of sequence segments are of substantially identical length.

27. A computer-readable medium whose contents cause a computer system to perform a method for forming a data repository of sequence segments to facilitate detection of apparent mutations in a target nucleic acid sequence, the computer system having a server program and a client program with functions for invocation by performing the steps of:
accessing a first plurality of sequence segments associated with a reference nucleic acid sequence, each of the first plurality of sequence segments being unique relative to one another;
determining possible variations for at least some of the first plurality of sequence segments; and
storing the possible variations in the data repository for subsequent comparison with at least a portion of the target nucleic acid sequence to detect apparent mutations therein.

28. A computer for analyzing a target nucleic acid sequence, wherein the computer comprises:
(a) memory storing an instruction set and reference data related to a reference nucleic acid sequence, wherein the reference nucleic acid sequence includes a plurality of reference sequence segments; and
(b) a processor for running the instruction set, the processor being in communication with the memory, wherein the processor is operative to:
(i) access the reference data;
(ii) determine if a target sequence segment of the target nucleic acid sequence matches one of the plurality of reference sequence segments;
(iii) if the target sequence segment does not match, identify the target sequence segment as a non-matched target sequence segment;
(iv) generate at least one single error variant of the non-matched target sequence segment; and
(v) compare the at least one single error variant with the plurality of reference sequence segments for a match.


SEQUENCE LISTING (8 sequences)

SEQ ID NO: 1 - DNA, 10 bp, Artificial Sequence
  Description of Artificial Sequence: Synthetic oligonucleotide
atgctcatta

SEQ ID NO: 2 - DNA, 25 bp, Artificial Sequence
  Description of Artificial Sequence: Synthetic oligonucleotide
aaaaaaaaaaaaaaaaaaaaaaaac

SEQ ID NO: 3 - DNA, 25 bp, Artificial Sequence
  Description of Artificial Sequence: Synthetic oligonucleotide
aaaaaaaaaaaaaaaaaaaaaaaag

SEQ ID NO: 4 - DNA, 25 bp, Artificial Sequence
  Description of Artificial Sequence: Synthetic oligonucleotide
aaaaaaaaaaaaaaaaaaaaaaaat

SEQ ID NO: 5 - DNA, 25 bp, Artificial Sequence
  Description of Artificial Sequence: Synthetic oligonucleotide
ccccccccccccccccccccccccc

SEQ ID NO: 6 - DNA, 25 bp, Artificial Sequence
  Description of Artificial Sequence: Synthetic oligonucleotide
cccccccccccccccccccccccca

SEQ ID NO: 7 - DNA, 25 bp, Artificial Sequence
  Description of Artificial Sequence: Synthetic oligonucleotide
ccccccccccccccccccccccccg

SEQ ID NO: 8 - DNA, 25 bp, Artificial Sequence
  Description of Artificial Sequence: Synthetic oligonucleotide
cccccccccccccccccccccccct